(19) United States
(12) Patent Application Publication
(10) Pub. No.: US 2014/0102469 A1
(43) Pub. Date: Apr. 17, 2014

(54) METHODS AND COMPOSITIONS FOR BONDING SILICONE POLYMERS TO HAIR AND NAILS

(71) Applicants: William Gunter LOUDON, San Clemente, CA (US); Dean Vegas CHRISTAL, Santa Barbara, CA (US); William Jeffrey BERTRAND, Ventura, CA (US)

(72) Inventors: William Gunter LOUDON, San Clemente, CA (US); Dean Vegas CHRISTAL, Santa Barbara, CA (US); William Jeffrey BERTRAND, Ventura, CA (US)

(73) Assignee: LIQWD, INC., Santa Barbara, CA (US)

(21) Appl. No.: 13/650,713

(22) Filed: Oct. 12, 2012

(51) Int. Cl. A61K 8/89; A61Q 3/00; A45D 29/00; A61K 8/18; A45D 7/00; A61K 8/898; A61Q 5/00

(52) U.S. Cl. 132/208; 424/70.12; 424/61; 132/202; 132/200

(57) ABSTRACT

Methods for covalently binding a silicone polymer to hair or nails using UV light, a catalyst, a reactive solution, and compositions related thereto. The methods can include synthesizing a photo-reactive silicone and bonding the photo-reactive silicone polymer to hair, nails, or keratin. The methods can also include applying a vinyl-containing silicone polymer and a catalyst, or an aldehyde functionalized silicone polymer and reactive composition, to covalently bond the silicone polymer to hair, nails, or keratin. An advantage of covalently bonding silicone polymer to hair or nails can be that the silicone polymer add strengthens and shine to hair and nails, but are more resistant to washings with soap and shampoo than non-covalently bound silicone polymers.

### CROSS-REFERENCE TO RELATED APPLICATIONS

This application is a continuation of PCT/US2012/054155, filed Sep. 7, 2012, which claims priority to U.S. Provisional Application No. 61/546,806, filed on Oct. 13, 2011, and U.S. Provisional Application No. 61/608,884, filed on Mar. 9, 2012.

### BACKGROUND

Traditional hair care treatments designed to reduce fraying and to add body, shine, and weight to hair have employed silicone oils that are formulated into a solution or emulsion for direct application to hair. Formulations of siloxane polymers (polysiloxanes), emulsifiers, and solvents have been used in shampoos, conditioners, styling compositions, and other hair care products to facilitate this direct application of silicones to hair. In particular, silicones are found in nearly every styling product. However, the positive benefits of the traditional direct application of silicones to hair are limited and short-lived. For example, Moroccan oil, which is one of the most popular products for styling, conditioning, and finishing hair, typically contains high percentages of cyclopentasiloxane, dimethicone, and cyclomethicone. Yet the shine and body gained by adding this product only lasts about an hour before it begins to dry out and its beneficial properties wear off. Further, all of the benefits of traditional products, like Moroccan oil, are lost after a single wash with soap and shampoo. This is because the surfactant properties of soaps and shampoos readily remove the siloxane oils applied to hair. Furthermore, simple mixing of different silicone species with the intent of combining specific desirable properties such as shine with softness generally dilutes the effects of each species.

The tendency for silicone and polysiloxane products to wash out of hair has forced consumers to apply conditioners and hair styling products to their hair on a daily basis. This daily reapplication of the same products to hair costs consumer valuable time, energy, and money. Further, it places a strain on the environment, because manufactures are continuously using resources to make products that are worn once and then washed down the drain.

Alternative methods for application of silicones and polysiloxanes, which provide the unique benefits of specific silicone polymers in a more durable or permanent formulation would provide significant improvements over conventional temporary applications. However, past attempts to increase retention of silicones and polysiloxanes to hair have left much to be desired.

For example, the “Brazilian hair straightening treatment” can involve the application of silicones and/or polysiloxanes to hair, then flat-ironing the hair to melt-bond the polymers to the hair. Although this application process transiently improves appearance, the hair itself is irreversibly damaged. The resultant damage can be so severe that when the polymer treatment wears out, the unprotected strand of hair is at risk of breaking off, possibly even resulting in baldness.

Nail treatments suffer from similar limitations of current methods of application. Consumers want finger and toe nails to have a luxurious, glamorous shine with colors of the consumer's choosing. However, the acrylic polymers typically used in nail polish are subject to chipping, breaking, and other forms of undesirable abrasion. In contrast, silicone polymers exhibit properties such as flexibility, toughness, and shine, which might replace or augment traditional nail treatments. Current modes for application of such silicone polymers have significant technical limitations such as their inability to resist washing off when subjected to simple soap and water.

There is a need for a method of treating hair and nails with silicone polymers, which is capable of providing stable benefits of silicone polymers, which resist spontaneous degradation and washing out with the simple application of soap and/or shampoo to clean the hair or nails. In addition, there is a need to imbue hair and nails with the glossy, protective properties of silicone polymers in a way that does not directly damage these fragile substrates, while providing protection from physical abuse such as abrasion and repeated washings. There is a need for a method of topically treating hair and nails with wash-resistant silicone polymers, wherein the method is accessible and supervised by hair care practitioners trained in the application of these polysiloxane formulations. There is also a need for a method, which allows a person to apply silicone polymers in a manner that selectively improves the retention of the silicone polymers to hair and nails.

### SUMMARY

Embodiments discussed herein relate to methods of bonding mixtures of silicone polymers to hair, nails, or keratin, processes for preparing the silicone polymers, and methods for using novel compositions containing these silicone polymers. The methods disclosed can advantageously be used to add softness, eliminate fraying, add body/weight to hair, add shine and improve coloration techniques for hair, nails, and keratin, and strengthen and protect nails and hair. The methods can maintain these beneficial properties even after one or more washings with the soap, shampoo, and other surfactant containing materials.

Provided herein is a method for bonding a silicone polymer to hair, or a nail comprising: providing at least one photo-reactive silicone polymer, bringing the at least one photo-reactive silicone polymer into contact with the hair or the nail, and applying UV light from a light source to the at least one photo-reactive silicone polymer in the presence of the hair or the nail to bond at least one silicone polymer to the hair or the nail, wherein the UV light has a wavelength of from about 200 nm to about 400 nm. The providing step can comprise reacting a compound a benzoyl benzoic acid with a silicone polymer having at least one amine group to form a photo-reactive silicone polymer. The at least one photo-reactive silicone polymer can have a molecular weight of from about 250 g/mol to about 75,000 g/mol, and can contain from about 0.5 to about 50 mole percent of a photo-reactive group. Alternatively, the at least one photo-reactive silicone polymer can have a molecular weight of from about 4,000 g/mol to about 60,000 g/mol, and can contain from about 1 to about 15 mole percent, including about 6 to about 7 mole percent, of the photo-reactive group, and the photo-reactive group is represented by Formula (I):

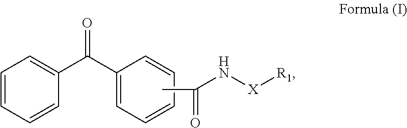

wherein the R₁ represents a silicone polymer, and X represents a linking group selected from the group consisting of: a covalent bond and a (C₁-C₁₀) hydrocarbyl group, optionally substituted with one or more of oxygen, nitrogen, sulfur, or silicon. Also, the at least one photo-reactive silicone polymer can have a molecular weight of from about 4,000 g/mol to about 50,000 g/mol, and the silicone polymer is a poly(dimethyl siloxane), and the photo-reactive group is selected from the group consisting of:

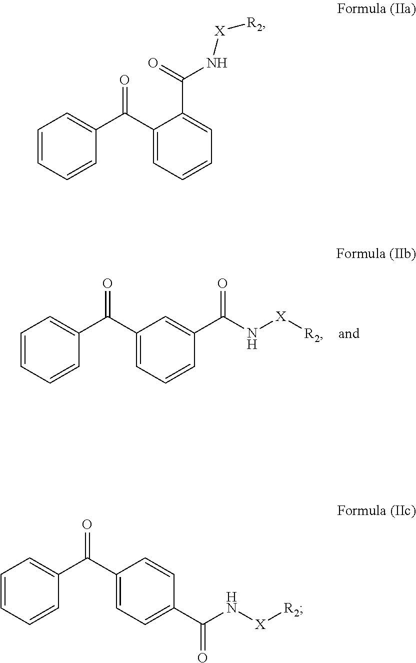

wherein R₂ represents the poly(dimethyl siloxane), and X represents a linking group selected from the group consisting of: a covalent bond and a (C₁-C₁₀) hydrocarbyl group, optionally substituted with one or more of oxygen, nitrogen, sulfur, or silicon. In an embodiment, the bringing step comprises: applying the at least one photo-reactive silicone polymer in a solution to the hair or the nail. The solution can comprise an aprotic solvent. The providing step can comprise: reacting a compound represented by Formula (III):

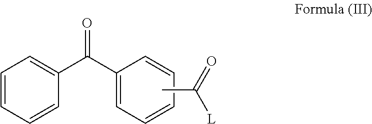

to produce the compound represented by Formula (I);

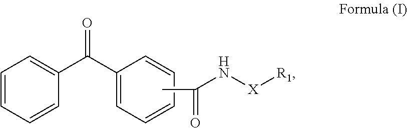

wherein R₁ represents a silicone polymer; X represents a linking group selected from the group consisting of: a covalent bond or a (C₁-C₁₀) hydrocarbyl group, optionally substituted with one or more of oxygen, nitrogen, sulfur, or silicon; and L represents Br or Cl. The method further can comprise a step of synthesizing a compound of Formula (III) by reacting a benzoyl benzoic acid with thionyl chloride, oxalyl chloride, or phosphorous tribromide to produce the compound of Formula (I). The providing step can comprise:

reacting a compound of Formula (IIIa);

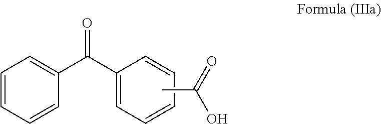

to form a compound of Formula (I);

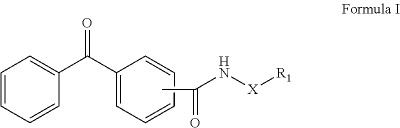

wherein R₁ represents a silicone polymer; and X represents a linking group selected from the group consisting of: a covalent bond and a (C₁-C₁₀) hydrocarbyl group, optionally substituted with one or more of oxygen, nitrogen, sulfur, or silicon. Optionally, before or after the bringing step, the hair can be subject to a permanent coloration, demi-permanent coloration, semi-permanent coloration, temporary hair color, or a straightening treatment, or combination thereof.

Provided herein is a method of applying a photo-reactive silicone polymer to hair or a nail comprising:

providing at least one photo-reactive silicone polymer, and

bringing the at least one photo-reactive silicone polymer into contact with the hair or the nail,

wherein the photo-reactive silicone polymer is capable of bonding to hair or a nail when the photo-reactive silicone polymer is exposed to UV light having a wavelength of from about 200 nm to about 400 nm from about 1 s to about 4 hours.

In another aspect, provided herein is a method of covalently bonding a silicone polymer to hair, or a nail comprises: providing at least one photo-reactive silicone polymer, bringing the at least one photo-reactive silicone polymer into contact with the hair or the nail, and applying UV light from a light source to the at least one photo-reactive silicone polymer in the presence of the hair or the nail to produce a covalent bond between at least one silicone polymer and the hair or the nail, wherein the UV light can have a wavelength of from about 200 nm to about 400 nm. The at least one photo-reactive silicone polymer can have a molecular weight of from about 250 g/mol to about 50,000 g/mol, and can contain from about 5 to about 15 mole percent of a photo-reactive group. The at least one photo-reactive silicone polymer can have a molecular weight of from about 4,000 g/mol to about 9,000 g/mol, can contain from about 6 to about 7 mole percent of the photo-reactive group, and the photo-reactive group is represented by Formula (I):

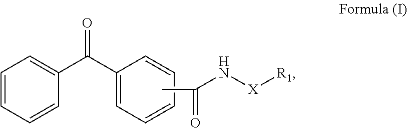

wherein the R₁ represents a silicone polymer, and X represents a linking group of a covalent bond or an alkyl group (CH₂)₁₋₁₀. Also, the at least one photo-reactive silicone polymer can have a molecular weight of from about 4,000 g/mol to about 6,000 g/mol, and the silicone polymer can be a poly(dimethyl siloxane), and the photo-reactive group can be represented by Formula (II):

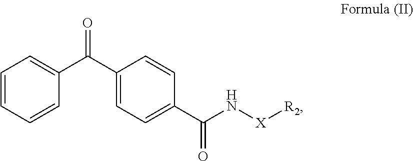

wherein R₂ represents the poly(dimethyl siloxane) and X represents a linking group of a covalent bond or an alkyl group (CH₂)₁₋₁₀. The light source can comprise at least one of a light emitting diode, a halogen bulb, a fluorescent bulb, or the sun. The bringing step can comprise: applying the at least one photo-reactive silicone polymer in a solution to the hair or the nail, wherein the solution comprises an aprotic solvent. The aprotic solvent can be tetrahydrofuran, a cyclomethicone, a dioxane, perfluorohexane, α,α,α-trifluorotoluene, pentane, hexane, cyclohexane, methylcyclohexane, decalin, carbon tetrachloride, freon-11, benzene, toluene, triethyl amine, carbon disulfide, diisopropyl ether, diethyl ether, t-butyl methyl ether, chloroform, ethyl acetate, 1,2-dimethoxyethane, 2-methoxyethyl ether, tetrahydrofuran, methylene chloride, pyridine, 2-butanone, acetone, hexamethylphosphoramide, N-methylpyrrolidinone, nitromethane, dimethylformamide, acetonitrile, sulfolane, dimethyl sulfoxide, propylene carbonate, or a mixture thereof. The providing step can comprise: synthesizing a compound represented by Formula (I) by reacting a compound represented by Formula (III):

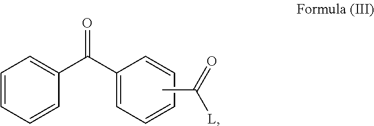

with an amine group attached to a silicone polymer to produce the compound represented by Formula (I)

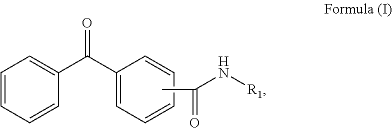

wherein R₁ represents a silicone polymer and L represents a leaving group. The leaving group can be chloride. In an embodiment, the synthesizing step can comprise reacting a benzoyl benzoic acid with thionyl chloride or oxalyl chloride to produce the compound of Formula (III). Additionally, before or after the bringing step, the hair can be subjected to a permanent coloration, demi-permanent coloration, semi-permanent coloration, temporary hair color, or a straightening treatment, or a combination thereof.

Another method contemplates, covalently bonding a silicone polymer to a hair or a nail comprising the steps of: providing at least one vinyl-containing silicone polymer, bringing the at least one vinyl-containing silicone polymer and a catalyst into contact with the hair or the nail, and forming a covalent bond between the at least one at least one vinyl-containing silicone polymer and the hair or the nail. The at least one vinyl-containing silicone polymer can have a molecular weight of from about 250 g/mol to about 50,000 g/mol, and wherein from about 4 to about 6 mole percent of a repeating unit of the at least one vinyl-containing silicone polymer contains a vinyl group. The molecular weight can range from about 4,000 g/mol to about 6,000 g/mol, and the vinyl groups are located at terminal ends of the at least one vinyl-containing silicone polymer. The at least one vinyl containing silicone polymer can be a poly(dimethyl siloxane). The catalyst can be a platinum catalyst, and ruthenium catalyst, rhodium catalyst, a tin catalyst, or a mixture thereof. During at least part of the step of forming a covalent bond, heated air or a heated surface can be applied to hair or nails from a heat source, wherein temperature of the heated air can be from 30-60° C., including 35-60° C. and 37-60° C., and wherein the heated surface can have a temperature of 35° C.-246° C., including 54° C.-246° C. or 130° F. to 475° F. Also, the bringing step can comprise: applying the at least one vinyl-containing silicone polymer in a solution to the hair or the nail, wherein the solution comprises an non-radical scavenging, aprotic solvent. The aprotic solvent can be tetrahydrofuran, a cyclomethicone, a dioxane, perfluorohexane, α,α,α-trifluorotoluene, pentane, hexane, cyclohexane, methylcyclohexane, decalin, carbon tetrachloride, freon-11, benzene, toluene, triethyl amine, carbon disulfide, diisopropyl ether, diethyl ether, t-butyl methyl ether, chloroform, ethyl acetate, 1,2-dimethoxyethane, 2-methoxyethyl ether, tetrahydrofuran, methylene chloride, pyridine, 2-butanone, acetone, hexamethylphosphoramide, N-methylpyrrolidinone, nitromethane, dimethylformamide, acetonitrile, sulfolane, dimethyl sulfoxide, propylene carbonate, or a mixture thereof. Optionally, before or after the bringing step, the hair can be subject to a permanent coloration, demi-permanent coloration, semi-permanent coloration, temporary hair color, a straightening treatment, or a combination thereof.

Yet another, a method of treating a hair to prevent or mitigate the effects of dryness, or flatness, and/or preventing color bleeding in hair can comprises: applying a composition comprising at least one photo-reactive silicone polymer to the hair. The at least one photo-reactive silicone polymer has a molecular weight of from about 250 g/mol to about 50,000 g/mol, and contains from about 5 to about 15 mole percent of a photo-reactive group. The at least one photo-reactive silicone polymer can have a molecular weight of from about 4,000 g/mol to about 9,000 g/mol, can contain from about 6 to about 7 mole percent of the photo-reactive group, and the photo-reactive group can be represented by Formula (I):

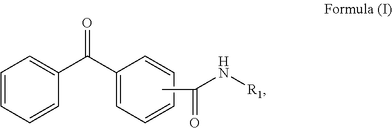

wherein the R₁ represents a silicone polymer. In an embodiment, the composition comprises: the at least one photo-reactive silicone polymer, wherein the solution comprises an aprotic solvent.

Also contemplated is a method of treating a hair to prevent or mitigate the effects of dryness, or flatness, and/or preventing color bleeding in a hair comprising: applying a composition comprising at least one vinyl-containing silicone polymer to the hair. The at least one vinyl-containing silicone polymer can have a molecular weight of from about 250 g/mol to about 50,000 g/mol, and wherein from about 4 to about 6 mole percent of a repeating unit of the at least one vinyl-containing silicone polymer can contain a vinyl group. The molecular weight can be from about 4,000 g/mol to about 6,000 g/mol, and the vinyl groups can be located at terminal ends of the at least one vinyl-containing silicone polymer. The at least one vinyl containing silicone polymer can be a poly(dimethyl siloxane). The catalyst can be a platinum catalyst, a ruthenium catalyst, a rhodium catalyst, a tin catalyst, or a mixture thereof.

Also provided is a method of covalently bonding a silicone polymer to hair, or a nail is disclosed comprising: providing at least one photo-reactive silicone polymer, bringing the at least one photo-reactive silicone polymer into contact with a hair or a nail, and applying UV light from a light source to the at least one photo-reactive silicone polymer in the presence of the hair or the nail to produce a covalent bond between at least one silicone polymer and the hair or the nail, wherein the UV light has a wavelength of from about 200 nm to about 400 nm, wherein the at least one photo-reactive silicone polymer has a molecular weight of from about 250 g/mol to about 50,000 g/mol, and contains from about 5 to about 15 mole percent of a photo-reactive group, wherein the photo-reactive group is represented by Formula (V):

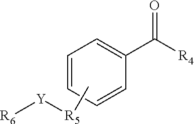

R₄ is selected from a (C₁-C₁₀) hydrocarbyl group;

R₅ is selected from the group consisting of: —N—(C═S)—N—; —(N—H)—; and —N═S(—H)—N—; —(C═O)—O—; —O—(C═O)—; and a (C₁-C₆) hydrocarbyl group, wherein the hydrocarbyl group is optionally substituted with one or more oxygen atoms, or hydroxyl groups; and

R₆ represents the silicone polymer; and

Y represents a linking group of a covalent bond or an alkyl group (CH₂)₁₋₁₀.

Another method of covalently bonding a silicone polymer to hair or nails comprises: providing at least one aldehyde functionalized silicone polymer; bringing the at least one aldehyde functionalized silicone polymer and a reactive composition into contact with hair or nails; forming a covalent bond between the at least one aldehyde functionalized silicone polymer and the hair or the nails. The reactive composition can be a solution comprising water and at least one acid. The solution has a pH of about 4 to about 9. During at least part of the step of forming a covalent bond, heated air or a heated surface is applied to the hair or nails from a heat source. The temperature of the heated air by the heat source is from about 30° C. to about 60° C., including from about 35° C. to about 60° C. and about 37° C. to about 60° C., and heated surface can have a temperature of 35° C.-246° C., including 54° C.-246° C. or 130° F. to 475° F. The at least one aldehyde functionalized silicone polymer has a molecular weight of from about 250 g/mol to about 50,000 g/mol, and contains from about 5 to about 15 mole percent of an aldehyde group. The at least one aldehyde functionalized silicone polymer has a molecular weight of from about 4,000 g/mol to about 9,000 g/mol, contains from about 6 to about 7 mole percent of the aldehyde group. Another method alters the providing step by providing an amine functionalized silicone polymer and a compound having at least two aldehyde groups; and reacting the amine functionalized silicone polymer with a least one aldehyde group of the compound having a least two aldehyde groups to form the at least one aldehyde functionalized silicone polymer. The compound having at least two aldehyde groups is a compound of Formula (IV):

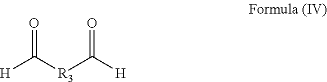

wherein R₃ is a (C₁-C₁₂) hydrocarbyl group, and wherein the hydrocarbyl group optionally includes at least one oxygen atom. The compound having at least two aldehyde groups can be selected from the group consisting of: malonic dialdehyde, succinic dialdehyde, glutaraldehyde, adipaldehyde, 3-methyl glutaraldehyde, propyladipaldehyde, phthalic dialdehyde, terephthaldehyde, and malonic dialdehyde.

The use of any of a photo-reactive silicone polymer of Formulas (I), (II), (IIa), and/or (IV), as described above, for a preparation to treat hair to prevent or mitigate the effects of dryness, or flatness, and/or preventing color bleeding in a hair.

The use of any of a photo-reactive silicone polymer of Formulas (I), (II), (IIa), (IIb),(IIc) and/or (IV), as described above, for a preparation to treat hair, wherein before or after the bringing step, the hair is subject to a permanent coloration, demi-permanent coloration, semi-permanent coloration, temporary hair color, or a straightening treatment or combination thereof.

It is to be understood that the foregoing general description and the following detailed description are exemplary and are intended to provide further explanation of the detailed compounds, compositions, and methods discussed herein.

### BRIEF DESCRIPTION OF DRAWINGS

Contained herein is at least one photograph executed in color. Copies of this patent or patent application publication with color photograph will be provided by the office upon request and payment of the necessary fee.

FIG. 1: A color photograph comparing two hair samples, wherein each was treated with a photo-active silicone polymer and washed. The hair sample on the left was treated with UV for thirty minutes prior to washing with shampoo and water. In contrast, the hair sample on the right was not treated with UV prior to washing with shampoo and water.

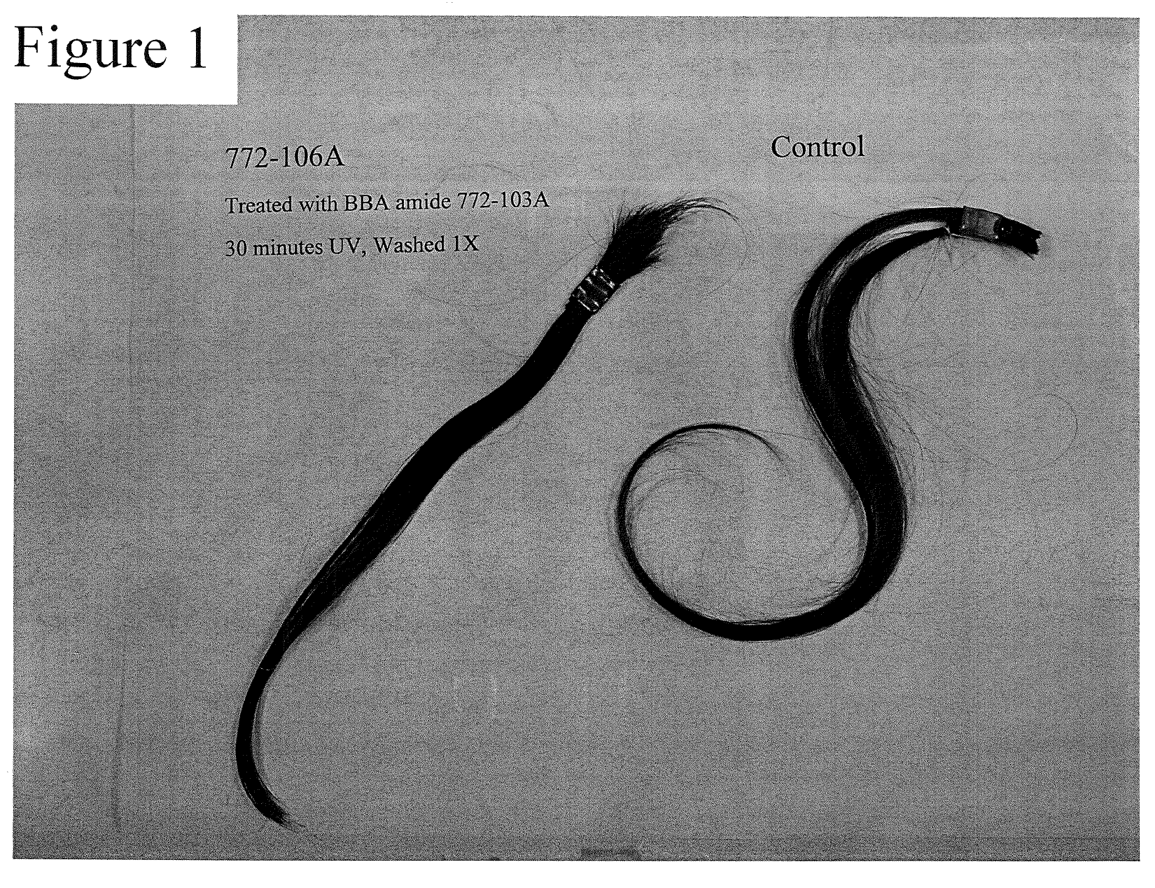

FIG. 2: A color photograph comparing two hair samples wherein each was treated with a photo-active silicone polymer and washed twice with shampoo and water. The hair sample on the left was treated with UV for thirty minutes prior to the washings. In contrast, the hair sample on the right was not treated with UV prior to the washings with shampoo and water.

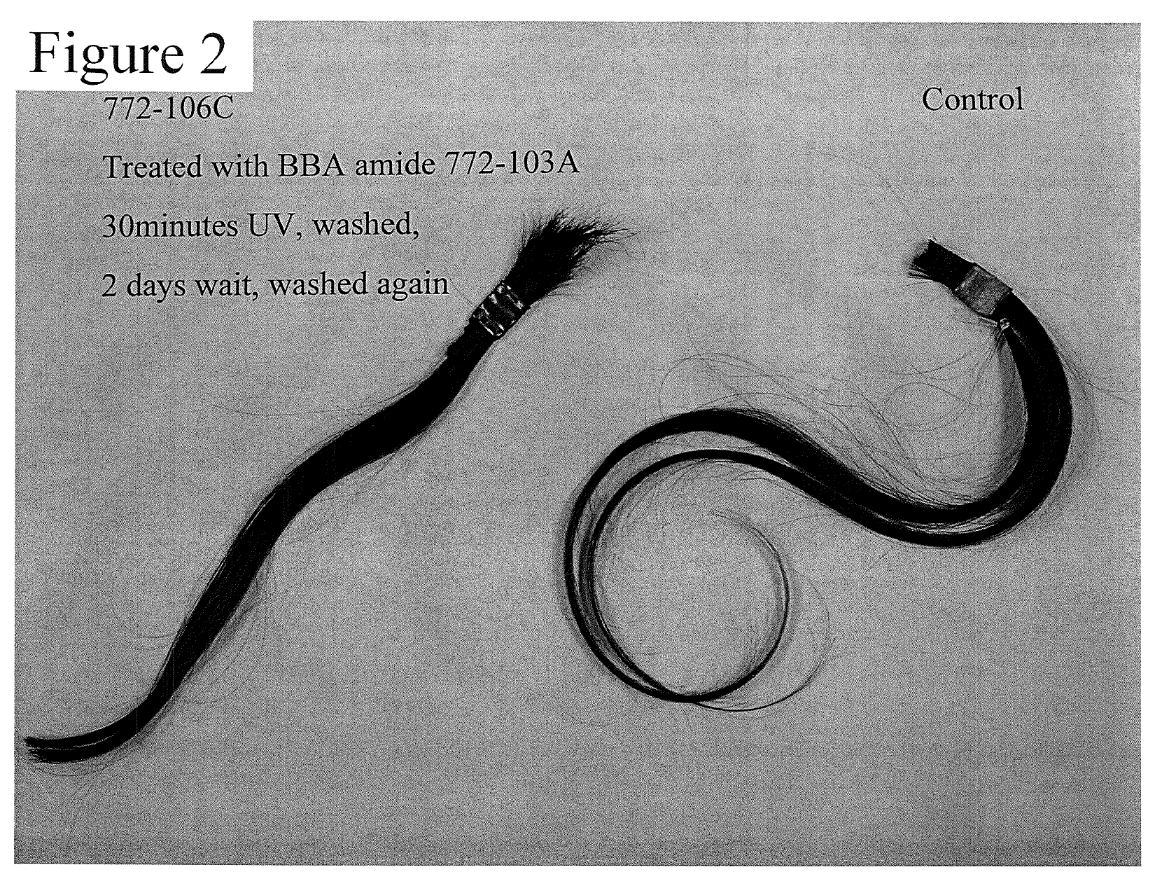

The photographs are exemplary only, and should not be construed as limiting the claims and embodiments provided herein.

### DETAILED DESCRIPTION

The following definitions apply throughout the specification, unless otherwise noted.

The term “silicone” means a polymer which has a repeating unit that consists of the elements silicon, carbon, hydrogen, and oxygen. Silicones can be modified with functional groups that contain additional elements. For example, a silicone polymer can be modified with an amine group, which would include nitrogen.

The term “polymer” means a molecule having at least one repeating unit, wherein the molecule has a molecular weight of at least 200 g/mol.

The term “about” means that the number being described can deviate by plus or minus twenty percent of the number. For example, “about 250 g/mol” means from 200-300 g/mol. However, when referring to a range the term “about” refers to twenty percent less than lower endpoint and twenty percent higher than upper endpoint. For example, “about 250 to about 50,000” means 250-60,000 g/mol.

The term “photo-reactive” refers to a molecule or chemical group which is capable of undergoing a chemical transformation to form a reactive group, such as a free radical, when irradiated with electromagnetic radiation. When activated, this group is capable of chemically reacting with other chemical groups, resulting in the creation of a new stable, covalent bond or cross-linkage.

The term “siloxane” refers to a silicone polymer having a repeating unit of

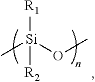

wherein “n” represents the number of times the repeating unit is repeated, and R₁ and R₂ can be the same or different. Further, the repeating unit, including R₁ and R₂, consist of the elements Si, O, C, and H. Silioxanes can be modified with additional functional groups that contain additional elements. For example, a siloxane be modified by a functional group, such as an alkyl amine, which includes nitrogen.

The term “polydimethyl siloxane” or “poly (dimethyl siloxane)” means a silicone or siloxane having the following structure

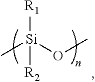

wherein “n” represents the number of times the repeating unit is repeated, and wherein R₁ and R₂ represent methyl groups. Poly(dimethyl siloxane)s can be abbreviated as “PDMS.” Polydimethyl siloxanes can be modified with additional functional groups that contain additional elements. For example, a polydimethyl siloxane can be modified by a functional group, such as an alkyl amine, which includes nitrogen.

The term “bleaching” refers to any chemical process, which removes at least part of the natural or synthetic color from hair. For example, bleaching agents, such as hydrogen peroxide and ammoniums hydroxide, can be used to removed natural or synthetic hair colors.

The term “permanent coloration,” refers to any process which bonds a dye to the cuticle of hair, and uses ammonia to expose the cuticle of hair. Permanent coloration bonds dye to the cortex of the hair and not to the cuticle. The cuticle is the outside of the hair. Ammonia lifts the cuticle to expose the cortex where the dye is bound.

The term “demi-permanent” refers to any process which bonds a dye to in and under the cuticle of hair, and does not use ammonia.

The term “semi-permanent” refers to any process which bonds dye to the cuticle of the hair, but does not bind dye to the cortex of hair.

The term “temporary hair color” refers to any process that deposits color on the cuticle of the hair but does not bond to the cuticle. This typically washes off with one shampoo.

The term “straightening treatment” refers to a process of breaking the hydrogen disulfide bonds of hair, or preventing or reducing the amount of hydrogen disulfide bonds capable of forming in hair.

The phrase “preventing color bleeding” refers to the prevention or reduction of the tendency of a cuticle of hair from opening and allowing color to be removed. The prevention or reduction of the opening of hair cuticles can extend the color lifetime of a color treatment, including permanent coloration, demi-permanent coloration, semi-permanent coloration, and temporary hair color, to diminish the amount of color lost during washings.

The term “vinyl-containing” refers to a molecule or chemical group containing at least one vinyl group.

The term “aprotic solvent” means a solvent that does not have an acidic hydrogen.

The term “nail” means a fingernail or toenail of a human or animal.

All measurements are in terms of metric units, unless otherwise noted.

An alternative spelling for “benzoyl benzoic acid” is “benzoil benzoic acid”, which means

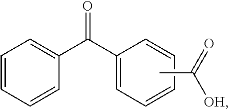

wherein the carboxylic acid can be substituted for any hydrogen on the aromatic ring. An aryl ring having a substitutent connected to the center of the ring rather than a carbon atom, indicates that the substitutent can be substituted for a hydrogen at any carbon on the aryl ring.

The term “hair” refers to one or more than one strand of hair, as well as the natural components of hair, such as oil from a body.

Unless otherwise noted, the terms “bonding”, “bond”, and “bonds,” when referring to bonding of a polymer to hair, means any manner of adhering the polymer to hair, including forming a covalent bond, cross-linking, and/or entanglement. For example, a polymer can be bound to hair by the formation of at least one covalent bond between the polymer and the hair. In addition, the polymer may form crosslinks around and among the strands of hair, such that the cross-linked polymer creates an interpenetrating network with the hair and resists removal during washing. Also, the polymer may become entangled in the hair such that the polymer resists removal during washing. Without wishing to be constrained by any particular theory, it is believed the methods provided herein bond a polymer to hair through one or more of covalent bonding, cross-linking, entanglement, and/or formation of an interpenetrating network.

The term “hydrocarbyl” refers to a moiety comprising only hydrogen and carbon atoms, unless otherwise explicitly stated. For example, a hydrocarbyl optionally substituted with one or more oxygen atoms includes an ester group, such as —(CH₂—CH₂)—O—CH₃. Further, a hydrocarbyl optionally substituted with one or more oxygen atoms includes an alcohol group, such as —C—(CH₃)₂—OH. A hydrocarbyl can form a non-aromatic, such as cyclohexane, or an aromatic ring, such as a substituted or unsubstituted (C₆-C₁₀)aryl group. Examples of hydrocarbyl groups can include phenyl and cyclohexyl groups. In addition, a hydrocarbyl group that is substituted by one or more oxygen atoms can include alkoxy groups and alkoxyalkyl groups, such as —O—CH₂—CH₃ or —CH₂—O—CH₃, respectively. Examples of a hydrocarbyl group substituted by one or more oxygen atoms can include —(C—OH)—(CH₃)₂ and —(C—OH)—(O—CH₃)₂.

It is understood that for the following description the term “siloxane” can be substituted for the term silicone, because a siloxane is a silicone. Further, the term poly(dimethyl siloxane) can be substituted for the term silicone or siloxane, because a poly(dimethyl siloxane) is a siloxane and a silicone.

While performing research in the field of personal care products and methods, it was discovered that the use of silicones can add weight and body to hair and/or prevent hair fraying. The silicones could be added to hair or coated onto nail, but the advantageous properties of the silicone were quickly lost after washing, especially when washing the hair or nail with a surfactant containing solution. Mixtures of different silicones can lose or exhibit diluted desirable qualities appreciated in the unmixed starting materials. Disclosed herein are the newly identified processes and products that bond silicones to hair, nail, and/or keratin to extend the beneficial effects of silicones for personal care products and animal care products.

Provided herein is a method of bonding a silicone polymer to hair or a nail comprising: providing at least one photo-reactive silicone polymer, bringing the at least one photo-reactive modified silicone polymer into contact with the hair or nail, applying UV light from a UV light source to the at least one photo-reactive silicone polymer in the presence of the hair or the nail to bond the at least one silicone polymer and the hair or the nail, wherein the utilized UV light has a wavelength of from about 200 nm to about 400 nm, including from about 230 nm to about 400 nm.

The providing step is not particularly limited so long as a photo-reactive silicone polymer is obtained. The photo-reactive silicone polymer can be obtained from one or more synthesis steps, or purchased or otherwise procured.

The providing step comprise at least two steps: a first synthesizing sub-step and a second synthesizing sub-step. The first synthesizing step can include a step of synthesizing a compound of Formula (III) structure by reacting a benzoyl benzoic acid with thionyl chloride, oxalyl chloride, or phosphorous tribromide to produce the compound of Formula (III). The benzoyl benzoic acid can be at least one of 2-benzoyl benzoic acid, 3-benzoyl benzoic acid, and 4-benzoyl benzoic acid, and can react with thionyl chloride, oxalyl chloride to form at least one of 2-benzoylbenzoyl chloride, 3-benzoylbenzoyl chloride, or 4-benzoylbenzoyl chloride, respectively. Similarly, the benzoyl benzoic acid can be at least one of 2-benzoyl benzoic acid, 3-benzoyl benzoic acid, and 4-benzoyl benzoic acid, and can react with phosphorous tribromide to form at least one of 2-benzoylbenzoyl bromide, 3-benzoylbenzoyl bromide, or 4-benzoylbenzoyl bromide, respectively.

The second synthesizing sub-step comprises reacting a compound represented by Formula (III):

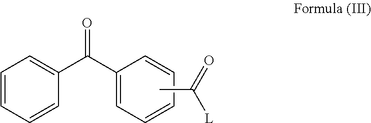

to produce the compound represented by Formula (I)

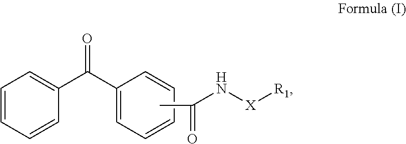

wherein R₁ represents a silicone polymer; X represents a linking group selected from the group consisting of: a covalent bond and a (C₁-C₁₀) hydrocarbyl group, optionally substituted with one or more of oxygen, nitrogen, sulfur, or silicon; and L represents Br or Cl. During the second synthesizing sub-step, a base can be present to facilitate the displacement of the leaving group. X can represent a linking group selected from the group of a covalent bond and a (C₁-C₁ₒ) alkyl group, including (C₁-C₆) alkyl group.

Alternatively, the providing step of the method can comprise reacting a compound of Formula (IIIa);

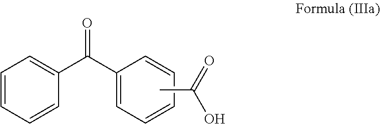

to form a compound of Formula (I);

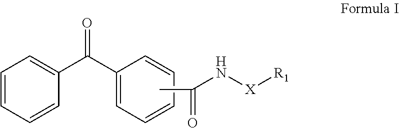

wherein R₁ represents a silicone polymer; and X represents a linking group selected from the group consisting of: a covalent bond and a (C₁-C₁₀) hydrocarbyl group, optionally substituted with one or more of oxygen, nitrogen, sulfur, or silicon. “X” can also be a linking group selected from the group of a covalent bond and a (C₁-C₁₀) alkyl group, including (C₁-C₆) alkyl group.

The reaction of Formula IIIa to form Formula I can be facilitated by the presence of a carbodiimide. The carbodiimide can include at least one of dicyclohexylcarbodiimide (DCC), (N-(3-dimethylaminopropyl)-N′-ethylcarbodiimide) (EDC), and diisopropylcarbodiimide (DIC). This reaction may be further facilitated by the presence of water and a base, such a piperidine.

The reaction can proceed with without the presence of a carbodiimide, by heating the compounds until an amide linkage forms from the acid and amine.

The second synthesizing sub-step can include a silicone polymer having at least one amine group can be reacted with a photo-reactive group, as represented by Formula (III)

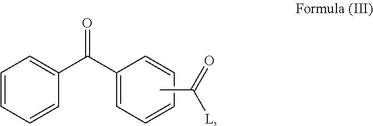

to form at least one of a compound selected from the group consisting of:

### Formula (IIa):

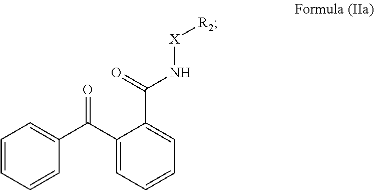

### Formula (IIb)

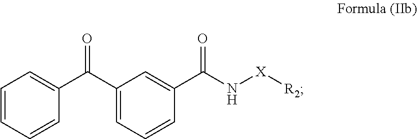

### and Formula (IIc)

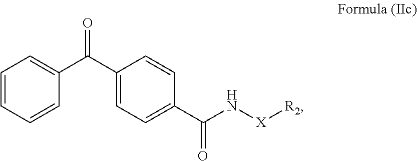

wherein R₂ represents a poly(dimethyl siloxane) and X represents a linking or spacer group selected from the group consisting of: a covalent bond or a (C₁-C₁₀) hydrocarbyl group, optionally substituted with one or more of oxygen, nitrogen, sulfur, or silicon, including a branched or unbranched (C₁-C₁₀) alkyl group, including an alkyl represented by —(CH₂)₃—.

The molecular weight of the at least one photo-reactive silicone polymer is not particularly limited, so long as the molecular weight of the silicone polymer allows for weight and body to be added to hair and/or to reduce fraying, add softness, and/or shine. It has been found that lower molecular weights can increase the shininess of hair, whereas high molecular weights can improve the coating properties of the silicone polymer on hair. For example, the molecular weight of the at least one photo-reactive silicone polymer can be about 500 to about 75,000 g/mol, including about 500 to about 60,000 g/mol, including about 4,000 to about 50,000 g/mol. Further, the silicone polymer can be branched or unbranched.

The photo-reactive silicone polymer can contain a photo-reactive group. The identity of the photo-reactive group is not particularly limited so long as the photo-reactive group can be irradiated with electromagnetic radiation to produce a reactive compound capable of bonding to hair, nails, or keratin. The photo-reactive group can form a free radical when irradiated with UV light from 200 to 400 nm, including 230 to 400 nm. The photo-reactive group can be an amide of benzyl benzoic acid, including an amide of 2-benzoyl benzoic acid, 3-benzoyl benzoic acid, or 4-benzoyl benzoic acid.

The photo-reactive silicone polymer can contain from about 0.5 to about 50 mole percent of a photo-reactive group relative to the moles of the repeating unit. The photo-reactive silicone polymer can contain from about 1 to about 15 mole percent of the photo-reactive group, including 5-10 mole percent, including 6-7 mole percent. An advantage of a higher mole percentage of photo-reactive groups can be that the photo-reactive silicone polymers can form more covalent bonds to the hair or nails and/or more cross-links, which increase the adhesive strength and/or its ability to resist repeated washings. However, the silicone polymer over cross-links if the mole percentage of photo-reactive groups becomes too high. Over cross-linking can lead to the formation of a silicone rubber that is not bound to the hair. Further, the presence of silicone rubber in hair can produce an undesirable look and/or feel for hair and nails.

The bringing step is not particularly limited so long as the at least one photo-reactive silicone polymer makes contact with the hair or the nail. The at least one photo-reactive silicone polymer can be brought into contact as a component of a liquid or gel formulation. The liquid or gel formulation can be applied using a sub-step of spraying, coating, painting, squirting, pouring, brushing, smearing, and other application techniques known in the art. The bringing step, a liquid or gel formulation can comprise a photo-reactive silicone polymer in a solution, mixture, or emulsion. In an embodiment of the solution, the solution, mixture, or emulsion includes an aprotic solvent, such as cyclomethicones, 1,4-dioxane, tetrahydrofuran, perfluorohexane, α,α,α-trifluorotoluene, pentane, hexane, cyclohexane, methylcyclohexane, decalin, carbon tetrachloride, freon-11, benzene, toluene, triethyl amine, carbon disulfide, diisopropyl ether, diethyl ether, t-butyl methyl ether, chloroform, ethyl acetate, 1,2-dimethoxyethane, 2-methoxyethyl ether, tetrahydrofuran, methylene chloride, pyridine, 2-butanone, acetone, hexamethylphosphoramide, N-methylpyrrolidinone, nitromethane, dimethylformamide, acetonitrile, sulfolane, dimethyl sulfoxide, propylene carbonate, or a mixture thereof. The identity of the aprotic solvent is not particularly limited, so long as the aprotic solvent is polar enough to dissolve the silicone polymer used and does not contain an acidic hydrogen. However, some aprotic solvents are more desirable than others due to concerns about safety or undesirable odors.

The applying UV step is not particularly limited so long as the photo-reactive silicone polymer can be bound to the hair or nails. The light source is not particularly limited so long as the light source can provide UV light in and/or across the range of from about 200 nm to about 400 nm, including 230 nm to 400 nm. The light source can include at least a mercury bulb, halide bulb, or even sunlight exposure for activation. The light source can be located in, or part, of a device, including a hand held device, or a tanning bed. The duration of applying the UV step is not particularly limited so long as at least one siloxane polymer bonds to a strand of hair. The duration of the UV step depends on the radiance (watts per square meter) of the step and the reactivity of the photo-active group. An advantage of a longer UV application step can be that stronger adhesion. However, if the UV application step is too long, the UV application step can irritate the skin and/or the individual may find the treatment time inconvenient. Development of new photo-active groups may significantly alter these parameters.

Additionally, before or after the bringing step, the hair can be subject to a hair treatment. Examples of hair treatments include: oil treatments, shampooing, detangling treatments, scalp treatments, shine treatments, dandruff treatments, curling treatments, straightening treatments, permanent hair treatments, temporary and permanent coloration treatments, and combinations thereof. An advantage of combining an embodiment of the method is the resistance of the polymer to washing out can extend the benefits of any of these hair treatments. For example, an advantage of the methods disclosed can be that the silicone polymer bound to the hair protects the cuticle of the hair from surfactants, which can remove coloring agents such as dyes. By protecting the cuticle of hair from surfactants, the color from, for example, a permanent or temporary coloring treatment can be preserved even after multiple washings with shampoo and soap. Another advantage of the embodiment of the method can be that the addition of body or weight to hair facilitates the straightening of hair, or prevents entanglement and frizzing. Another advantage of the methods disclosed is that they can allow for the application and retention of a desired mixture, or alternatively, the sequential application of individual silicone species to produce additive enrichments. The application of a mixture of silicone polymers or adding silicone polymer in a sequence can produce the same or different desirable cosmetic effects from those found in conventional application techniques. For example, amodimethicones are excellent conditioners, providing improved compatibility, improved feel and softness, and diminish fraying. Phenyltrimethicones can be used in reflection-enhancing and color-correcting hair products, where they increase shine, glossiness, and color. When combined in the same formulation and applied through a conventional manner to hair, amodimethicone and phenyltrimethicone interact and dilute each other, making it difficult to achieve both high shine and excellent conditioning in the same product. Serial binding applications of each separate silicone polymer can escape this negative interaction allowing for maximal benefits of their individual qualities. Another advantage of an embodiment of the method can be that binding silicones to hair, skin, and nails may add structural support to the protein structure of these appendages, thereby providing them with some enhanced protection from physical, chemical, and thermal damage. The step of bringing the photo-reactive polymer into contact with hair can be performed from about 1 second to about 72 hours before or after a treatment.

The method can be applied to any subject with hair or nails, including humans, but can also include animals having hair or nails. The method can also be applied to eyebrows and eyelashes. Further, the method can be applied to non-living subjects, including hair in wigs or clothing, such as coats, made from human or animal hair; artificial nails (e.g., acrylic nails); and implanted hair, artificial hair (e.g. polyesters, nylons, acrylic polymers), so long as the photoreactive polymer is capable of binding the silicone polymer to artificial nails and hair.

The photo-reactive group can differ from that above. For example, the at least one photo-reactive silicone polymer can have at least one photo-reactive group attached to the silicone polymer. The identity of the photo-reactive silicone is not particularly limited, so long as the photo-reactive groups is attached to the silicone polymer by, for example, a covalent bond, and the photo-reactive group is capable of forming a free radical upon irradiation. Irradiation in the form of UV light has a wavelength of from about 200 nm to about 400 nm and would generate at least one free radical from the photoreactive group to facilitate bonding the silicone polymer to hair. The method of bonding a silicone polymer to hair, or a nail comprises: providing at least one photo-reactive silicone polymer, bringing the at least one photo-reactive silicone polymer into contact with the hair or the nail, and applying UV light from a light source to the at least one photo-reactive silicone polymer in the presence of the hair or the nail to bond the at least one silicone polymer to the hair or the nail, wherein the UV light has a wavelength of from about 200 nm to about 400 nm, wherein the at least one photo-reactive silicone polymer has a molecular weight of from about 250 g/mol to about 75,000 g/mol, including 4,000-50,000 g/mol, including about and contains from about 0.5 to about 50 mole percent of a photo-reactive group, including 1-15 mole percent, and 6-7 mole percent of a photo-reactive group, wherein the photo-reactive group is represented by Formula (V):

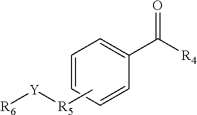

R₄ is selected from a (C₁-C₁₀) hydrocarbyl group;

R₅ is selected from the group consisting of: —N—(C—S)—N—; —(N—H)—; and —(N—H—; and —N═S(—H)—N—; —(C═O)—O—; —O—(C═O)—; and a (C₁-C₆) hydrocarbyl group, wherein the hydrocarbyl group is optionally substituted with one or more oxygen atoms, or hydroxyl groups; and R₆ represents the silicone polymer; and Y represents a linking group of a covalent bond or an alkyl group (CH₂)₁₋₁₀. In an embodiment, R₄ is selected from the group consisting of: phenyl, and a hydroxyl substituted cyclohexyl.

By way of examples, it understood the following photo-reactive molecules can be reacted with an amine or thiol group on a silicone polymer to form a photo-reactive silicone polymer: N-((2-pyridyldithio)ethyl)-4-azidosalicylamide; (4-azido-2,3,5,6-tetrafluorobenzoic acid, succinimidyl ester); 1-(4-benzoylphenyl)-1H-pyrrole-2,5-dione; benzophenone-4-isothiocyanate; 4-benzoylbenzoic acid, succinimidyl ester; and (N-((2-pyridyldithio)ethyl)-4-azidosalicylamide). Each of these molecules is known in the art and can be purchased from, e.g., Molecular Probes® (Grand Island, N.Y.). Further, the following photo-reactive molecules can also be attached to a silicone polymer by methods known in the art:

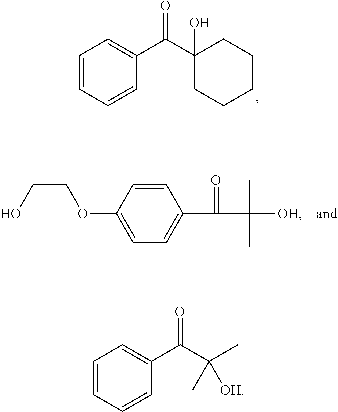 [SMILES: O=C(c1ccccc1)C1(O)CCCCC1 ; CC(C)(O)C(=O)c1ccc(OCCO)cc1 ; "and" ; CC(C)(O)C(=O)c1ccccc1]

These molecules are known in the art and are commercially available from, e.g., Ciba Specialty Chemicals (Tarrytown, N.Y.). It is contemplated that such photo-reactive molecules can be bound to silicone polymer having at least one alcohol, carboxylic acid, and/or amine through a variety of reactions. For example, a silicone polymer having an alcohol group can be reacted with one of the above compounds in the presence of a cross-coupling agent, such as, dicyclohexylcarbodiimide (DCC) in an appropriate solvent to produce a photo-reactive silicone polymer.

In an embodiment, a method of covalently bonding a silicone polymer to hair or a nail comprises: providing at least one photo-reactive silicone polymer, bringing the at least one photo-reactive modified silicone polymer into contact with hair or nail, applying UV light from a UV light source to the at least one photo-reactive silicone polymer in the presence of the hair or the nail to produce a covalent bond between at least one silicone polymer and the hair or the nail, wherein the utilized UV light has a wavelength of from about 200 nm to about 400 nm, including from about 230 nm to about 400 nm.

The providing step is not particularly limited so long as a photo-reactive silicone polymer is obtained. In an embodiment, the photo-reactive silicone polymer can be obtained from a synthesizing step or purchased.

The synthesizing step can include one or more sub-steps and is not particularly limited so long as at least one photo-reactive silicone polymer is obtained. In an embodiment of a first synthesizing sub-step, a photo-reactive compound can be reacted, with another compound having a leaving group to form a photo-reactive compound having a leaving group, as represented by Formula III:

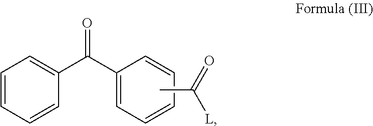

wherein L represents a leaving group. For example, in an embodiment, the first synthesizing sub-step includes reacting a benzoil benzoic acid with a thionyl chloride or oxalyl chloride to produce the compound of Formula (III). During the first synthesizing sub-step, a base can be present to facilitate the displacement of the leaving group.

In an embodiment of a second synthesizing sub-step, a silicone polymer having at least one amine group attached by a linking group can be reacted, with a photo-reactive group having a leaving group, as represented by Formula (III), to form a molecule represented by Formula (I):

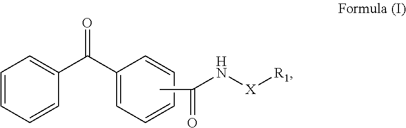

wherein R₁ represents a silicone polymer, and X represents a linking or spacer group of a covalent bond or an alkyl group (CH₂)₁₋₁₀, including an alkyl represented by (CH₂)₃. During the second synthesizing sub-step, a based can be present to facilitate of the formation of the amide group. The base for the first and second sub-steps is not particularly limited as long as it lowers the pH of the reaction. The base can be can be different or the same for each sub-step and can include diisopropylethylamine.

Further, in the second synthesizing sub-step, a silicone polymer having at least one amine group can be reacted with a photo-reactive group having a leaving group, as represented by Formula (III), to form a molecule represented by Formula (II):

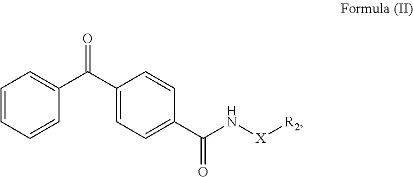

wherein R₂ represents a poly(dimethyl siloxane) and X represents a linking or spacer group of a covalent bond or an alkyl group (CH₂)₁₋₁₀, including an alkyl represented by (CH₂)₃.

A first synthesizing sub-step, 4-benzoil benzoic acid can react with thionyl chloride or oxalyl chloride to produce 4-benzoil benzoic acid chloride. In an embodiment of a second synthesizing sub-step, 4-benzoil benzoic acid chloride can react with a silicone polymer having at least one amine group to produce a photo-reactive silicone polymer. An example of a silicone polymer having at least one amine group is 6-7% aminopropylmethylsiloxane-dimethylsiloxane copolymer, which is commercially available as Gelest AMS-162 (Gelest, Inc., USA).

The at least one photo-reactive silicone polymer can have at least one photo-reactive group attached to the silicone polymer. The identity of the photo-reactive silicone is not particularly limited, so long as the photo-reactive groups is attached to the silicone polymer by, for example, a stable covalent bond, and the photo-reactive group is capable of forming a free radical upon irradiation. Irradiation in the form of UV light has a wavelength of from about 200 nm to about 400 nm and would generate at least one free radical from the photoreactive group to facilitate bonding the silicone polymer to hair. The method of covalently bonding a silicone polymer to hair, or a nail comprising: providing at least one photo-reactive silicone polymer, bringing the at least one photo-reactive silicone polymer into contact with the hair or the nail, and applying UV light from a light source to the at least one photo-reactive silicone polymer in the presence of the hair or the nail to produce a covalent bond between at least one silicone polymer and the hair or the nail, wherein the UV light has a wavelength of from about 200 nm to about 400 nm, wherein the at least one photo-reactive silicone polymer has a molecular weight of from about 250 g/mol to about 50,000 g/mol, and contains from about 5 to about 15 mole percent of a photo-reactive group, wherein the photo-reactive group is represented by Formula (V):

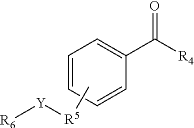

R₄ is selected from a (C₁-C₁₀) hydrocarbyl group;

R₅ is selected from the group consisting of: —N—(C═S)—N—; —(N—H)—; and —N═S(—H)—N—; —(C═O)—O—; —O—(C═O)—; and a (C₁-C₆) hydrocarbyl group, wherein the hydrocarbyl group is optionally substituted with one or more oxygen atoms, or hydroxyl groups; and

R₆ represents the silicone polymer; and

Y represents a linking group of a covalent bond or an alkyl group (CH₂)₁₋₁₀.

In an embodiment, R4 is selected from the group consisting of: phenyl, hydroxyl substituted cyclohexyl,

The term “hydrocarbyl” refers to any moiety comprising only hydrogen and carbon atoms, unless otherwise explicitly stated. For example, a hydrocarbyl optionally substituted with one or more oxygen atom includes an ester group, such as —(CH₂—CH₂)—O—CH₃. Further, a hydrocarbyl optionally substituted with one or more oxygen atom includes an alcohol group, such as —C—(CH₃)₂—OH. A hydrocarbyl can form a non-aromatic, such as cyclohexane, or an aromatic ring, such as a substituted or unsubstituted (C₆-C₁₀)aryl group. Examples of hydrocarbyl groups can include phenyl and cyclohexyl groups. In addition, a hydrocarbyl group that is substituted by one or more oxygen atoms can include alkyoxy groups and alkyloxyalkyl groups, such as —O—CH₂—CH₃ or —CH₂—O—CH₃, respectively. Examples of a hydrocarbyl group substituted by one or more oxygen atoms can include —(C—OH)—(CH₃)₂ and —(C—OH)—(O—CH₃)₂.

By way of examples, it understood the following photo-reactive molecules can be reacted with an amine or thiol group on a silicone polymer to form a photo-reactive silicone polymer: N-((2-pyridyldithio)ethyl)-4-azidosalicylamide; (4-azido-2,3,5,6-tetrafluorobenzoic acid, succinimidyl ester); 1-(4-benzoylphenyl)-1H-pyrrole-2,5-dione; benzophenone-4-isothiocyanate; 4-benzoylbenzoic acid, succinimidyl ester; and (N-((2-pyridyldithio)ethyl)-4-azidosalicylamide). Each of these molecules is known in the art and can be purchased from, e.g., Molecular Probes® (Grand Island, N.Y.). Further, the following photo-reactive molecules can also be attached to a silicone polymer by methods known in the art:

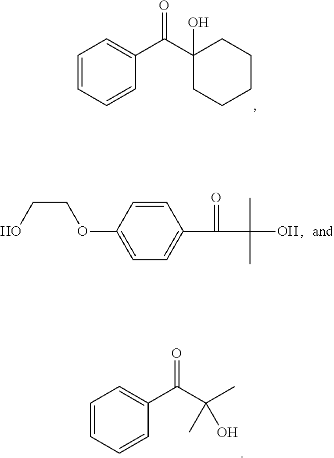 [SMILES: O=C(c1ccccc1)C1(O)CCCCC1 ; CC(C)(O)C(=O)c1ccc(OCCO)cc1 ; CC(C)(O)C(=O)c1ccccc1]

These molecules are known in the art and are commercially available from, e.g., Ciba Specialty Chemicals (Tarrytown, N.Y.). It is contemplated that such photo-reactive molecules can be bound to silicone polymer having at least one alcohol, carboxylic acid, and/or amine through a variety of reactions. For example, a silicone polymer having an alcohol group can be reacted with one of the above compounds in the presence of a cross-coupling agent, such as, dicyclohexylcarbodiimide (DCC) in an appropriate solvent to produce a photo-reactive silicone polymer.

In an embodiment, the molecular weight of the at least one photo-reactive silicone polymer is not particularly limited, so long as the molecular weight of the silicone polymer allows for weight and body to be added to hair and/or to reduce fraying, adds softness, and/or shine. It has been found that lower molecular weights can increase the shininess of hair, whereas high molecular weights can improve the coating properties of the silicone polymer on hair. In an embodiment, the molecular weight of the at least one photo-reactive silicone polymer can be 500 to 50,000 g/mol, including 500 to 9,000 g/mol, including 4,000 to 6,000 g/mol. Further, the silicone polymer can be branched or unbranched.

In an embodiment, the photo-reactive silicone polymer can contain a photo-reactive group. The identity of the photo-reactive group is not particularly limited so long as the photo-reactive group can be irradiated with electromagnetic radiation to produce a reactive compound capable of bonding to hair, nails, skin, or keratin. In an embodiment, the photo-reactive group can form a free radical when irradiated with UV light from 200 to 400 nm, including 230 to 400 nm. In an embodiment, the photo-reactive group can be an amide of benzyl benzoic acid, including an amide of 4-benzoyl benzoic acid.

In an embodiment, the photo-reactive silicone polymer can contain from about 5 to about 15 mole percent of a photo-reactive group relative to the moles of the repeating unit. The photo-reactive silicone polymer can contain from about 6 to about 7 mole percent of the photo-reactive group. An advantage of a higher mole percentage of photo-reactive group can be that the photo-reactive silicone polymers form more covalent bonds to the hair or nails, which increases the adhesive strength and/or its ability to resist repeated washings. However, the silicone polymer over cross-links if the mole percentage of photo-reactive groups becomes too high. Over cross-linking can lead to the formation of a silicone rubber that is not bound to the hair. Further, the presence of silicone rubber in hair can produce an undesirable look and/or feel for hair and nails.

In an embodiment, the bringing step is not particularly limited so long as the at least one photo-reactive silicone polymer makes contact with the hair or the nail. In an embodiment, the at least one photo-reactive silicone polymer can be brought into contact as a component of a liquid or gel formulation. In a further embodiment, the liquid or gel formulation can be applied using a sub-step of spraying, coating, painting, squirting, pouring, brushing, smearing, and other application techniques known in the art. In an embodiment, the liquid or gel formulation comprises a solution, mixture, or emulsion. In an embodiment of the solution, the solution, mixture, or emulsion include an aprotic solvent, such as cyclomethicones, 1,4-dioxane, tetrahydrofuran, perfluorohexane, α,α,α-trifluorotoluene, pentane, hexane, cyclohexane, methylcyclohexane, decalin, carbon tetrachloride, freon-11, benzene, toluene, triethyl amine, carbon disulfide, diisopropyl ether, diethyl ether, t-butyl methyl ether, chloroform, ethyl acetate, 1,2-dimethoxyethane, 2-methoxyethyl ether, tetrahydrofuran, methylene chloride, pyridine, 2-butanone, acetone, hexamethylphosphoramide, N-methylpyrrolidinone, nitromethane, dimethylformamide, acetonitrile, sulfolane, dimethyl sulfoxide, propylene carbonate, or a mixture thereof. The identity of the aprotic solvent is not particularly limited, so long as the aprotic solvent is polar enough to salivate the silicone polymer used and does not contain an acidic hydrogen. However, some aprotic solvents are more desirable than others due to concerns about safety or undesirable odors.

In an embodiment, the applying UV step is not particularly limited so long as the photo-reactive silicone polymer can be covalently bound to the hair or nails. In an embodiment, the light source is not particularly limited so long as the light source can provide UV light in or across the range of from about 200 nm to about 400 nm, including 230 nm to 400 nm. The light source can include at least a light emitting diode, a halogen bulb, and a fluorescent bulb, or even sunlight exposure for activation. The light source can be located in, or part, of a device, including a hand held device, or a tanning bed. The duration of the applying UV step is not particularly limited so long as at least one covalent bond can be formed linking the siloxane group to the hair strand. The duration of the UV step depends on the radiance (watts per square meter) of the step and the reactivity of the photo-active group. For example, with the photo-chemistry described herein, using a fluorescent bulb, the duration of the application step can range from 0.01 seconds to 24 hours, including 0.01 seconds to 1 hour, including 1 second to thirty minutes and any 0.01 second value there between. An advantage of a longer UV application step can be that more covalent bonds are formed. However, if the UV application step is too long, the UV application step can irritate the skin and/or the individual may find the treatment time inconvenient. Development of new photo-active groups may significantly alter these parameters.

In an embodiment, before or after the bringing step, the hair can be subject to a hair treatment. Examples of hair treatments include: oil treatments, shampooing, detangling treatments, scalp treatments, shine treatments, dandruff treatments, curling treatments, straightening treatments, permanent hair treatments, temporary and permanent coloration treatments, and combinations thereof. An advantage of combining an embodiment of the method is that the resistance of the polymer to washing out can extend the benefits of any of these hair treatments. For example, an advantage of the methods disclosed can be that the silicone polymer covalently bound to the hair protects the cuticle of the hair from surfactants, which can remove coloring agents such as dyes. By protecting the cuticle of hair from surfactants, the color from, for example, a permanent or temporary coloring treatment can be preserved even after multiple washings with shampoo and soap. Another advantage of an embodiment of the method can be that the addition of body or weight to hair facilitates the straightening of hair, or prevents entanglement and frizzing. Another advantage of a method disclosed is that it can allow for the covalent application of a desired mixture, or alternatively, the sequential application of individual silicone species to produce additive enrichments. The application of a mixture of silicone polymers or adding silicone polymer in a sequence can produce the same or different desirable cosmetic effects from those found in conventional application techniques. For example, amodimethicones are excellent conditioners, providing improved compatibility, improved feel and softness, and diminish fraying. Phenyltrimethicones can be used in reflection-enhancing and color-correcting hair products, where they increase shine, glossiness, and color. When combined in the same formulation and applied through a conventional manner to hair, amodimethicone and phenyltrimethicone interact and dilute each other, making it difficult to achieve both high shine and excellent conditioning in the same product. Serial covalent applications of each separate silicone polymer can escape this negative interaction allowing for maximal benefits of their individual qualities. Another advantage of an embodiment of the method can be that the covalent application of silicones to hair, skin, and nails may add structural support to the protein structure of these appendages, thereby providing them with some enhanced protection from physical, chemical, and thermal damage. The step of bringing can be performed from about 1 second to about 72 hours before or after a treatment.

In an embodiment, the method can be applied to any subject with hair or nails, including humans, but can also include animals having hair or nails. In an embodiment, the method can be applied to eyebrows and eyelashes. Further, the method can be applied to non-living subjects, including hair in wigs or clothing, such as coats, made from human or animal hair; artificial nails (e.g., acrylic nails); and artificial hair (e.g. polyesters, nylons, acrylic polymers), so long as the artificial nails and hair are capable of reacting covalently with a free radical to produce a covalent bond to silicone polymers.

In another embodiment, a method of covalently bonding a silicone polymer to a hair, skin, keratin, or a nail comprises: providing at least one vinyl-containing silicone polymer, bringing the at least one vinyl-containing silicone polymer and a catalyst into contact with the hair or the nail, and forming a covalent bond between the at least one at least one vinyl-containing silicone polymer and the hair or the nail.

The providing step is not particularly limited, so long as at least one vinyl-containing silicone polymer is obtained. In an embodiment, the providing step includes synthesizing at least one vinyl-containing silicone polymer or purchasing the at least one vinyl-containing silicone polymer.

The molecular weight of the at least one vinyl-containing silicone polymer is not particularly limited, so long as the molecular weight of the vinyl-containing silicone polymer allows for weight and body to be added to hair and/or reduces fraying. It has been found that lower molecular weights can increase the shininess of hair, whereas high molecular weights can improve the coating properties of the silicone polymer on hair. Thus, the molecular weight of the at least one vinyl-containing silicone polymer silicone polymer can be 500 to 50,000 g/mol, including 500 to 9,000 g/mol, including 4,000 to 6,000 g/mol. Further, the vinyl-containing silicone polymer can be branched or unbranched.

The at least one vinyl-containing silicone polymer can contain a vinyl group. The identity of the vinyl group is not particularly limited so long as the vinyl group can undergo a chemical reaction with hair, nails, and/or keratin to form a covalent bond. In an embodiment, the vinyl group can undergo a hydrosilylation in the presence of a catalyst and a suitable substrate, including hair, a nail, or keratin.

The number and position of the vinyl groups is not particularly limited, so long as the vinyl groups are capable of covalently bonding the silicone polymer to hair or nails. In an embodiment, the majority of the vinyl groups are located at terminal ends of the at least one vinyl-containing silicone polymer. In an embodiment, from about 4 to about 6 mole percent of a repeating unit of the at least one vinyl-containing silicone polymer contains a vinyl group. An advantage of a higher mole percentage of vinyl groups can be that the silicone polymers form more covalent bonds to the hair, nails, or keratin, which increases the adhesion and ability to resist removal upon repeated washings. However, if the mole percentage of vinyl groups goes too high, then the vinyl-group containing silicone polymer can cross-link with itself to form a silicone rubber. This silicone rubber in turn can produce an undesirable look or feel for hair and nails.

The bringing step is not particularly limited so long as the at least one vinyl-containing silicone polymer comes into contact with the hair or the nail. The bringing step is not particularly limited, so long as contact can be established between the at least one vinyl-containing silicone polymer, a catalyst, and hair or nails, wherein the catalyst can facilitate the formation of a covalent bond between the at least one vinyl-containing silicone polymer and the hair or the nail.

In an embodiment, the at least one vinyl-containing silicone polymer can be brought into contact as part of a liquid or gel formulation. The liquid or gel formulation can be applied using a sub-step of spraying, squirting, painting, coating, pouring, smearing, and other application techniques known in the art. In an embodiment, the liquid or gel formulation comprises a solvent, including cyclomethicones, 1,4-dioxane, tetrahydrofuran, or a mixture thereof. The identity of the solvent is not particularly limited, so long as the solvent is polar enough to dissolve the silicone polymer and a catalyst. The solvent can be an aprotic solvent, including tetrahydrofuran, a cyclomethicone, a dioxane, perfluorohexane, α,α,α-trifluorotoluene, pentane, hexane, cyclohexane, methylcyclohexane, decalin, carbon tetrachloride, freon-11, benzene, toluene, triethyl amine, carbon disulfide, diisopropyl ether, diethyl ether, t-butyl methyl ether, chloroform, ethyl acetate, 1,2-dimethoxyethane, 2-methoxyethyl ether, tetrahydrofuran, methylene chloride, pyridine, 2-butanone, acetone, hexamethylphosphoramide, N-methylpyrrolidinone, nitromethane, dimethylformamide, acetonitrile, sulfolane, dimethyl sulfoxide, propylene carbonate, or a mixture thereof. In an embodiment, the formulation does not contain a radical scavenger, which is any compound (other than oxygen) which tends to interfere with free radical reaction of vinyl groups. For example, bis-phenols and lactones are radical scavengers. A non-radical scavenging solvent is a solvent that does not interfere with free radical reaction of vinyl groups.

In a further aspect, the bringing step can include a catalyst. The catalyst can include a platinum catalyst, and ruthenium catalyst, rhodium catalyst, or a tin catalyst. The catalyst can be a hydrosilylation catalyst. The hydrosilylation catalyst can include pentamethylcyclopentadienyltris(acetonitrile)-ruthenium(II) hexafluorophosphate; chloroplatinic acid hydrate; platinum(0)-1,3-divinyl-1,1,3,3-tetramethyldisiloxane complex; bis(1,5-cyclooctadiene)rhodium(I) tetrafluoroborate hydrate; (bicyclo[2.2.1]hepta-2,5-diene)rhodium(I) chloride dimer, tris(triphenylphosphine)rhodium(I) chloride; benzenedichlororuthenium(II) dimer; dichloro(p-cymene)ruthenium(II) dimer; benzylidenebis(tricyclohexylphosphine)dichloro-ruthenium(II) (all of which are available from Aldrich, Missouri). Tin catalysts, such as SnCl₂, can be used to reduce costs.

During at least part of the step of forming a covalent bond, heated air or a heated surface can be applied to hair or nails from a heat source, wherein temperature of the heated air can be from 30-60° C., including 35-60° C. and 37-60° C., and wherein the heated surface can have a temperature of 35° C.-246° C., including 54° C.-246° C. or 130° F. to 475° F. The step of forming a covalent bond can include supplying heated air from a heat source, wherein the heated air can have a temperature from 30-60° C., including 35-60° C. and 37-60° C. The step of forming a covalent bond can include applying a heat source in the form of a hot surface, such as a flat iron or the like, to hair, wherein the temperature of the heated surface can be from 35° C.-246° C., including 54° C.-246° C. or 130° F. to 475° F. The formation of the covalent bond to hair or nails can proceed without the addition of hot air or a heated surface. However, supplying heated air or a heated surface can accelerate covalent bond formation, ensure the formation of as many covalent bonds as possible, and can be part of another treatment or larger process, such as drying hair after a straightening treatment. If the heated air temperature extends above 60° C., then the hot air can cause human and animal discomfort, and can damage the hair, skin, or nails. If the heated surface temperature exceeds 475° F., then the hair or nails can be damaged. However, if the temperature is too low, then the beneficial effects of applying heated air or a heated surface may not be observed.

In an embodiment, before or after the bringing step, the hair can be subject to a hair treatment. Examples of hair treatments include: oil treatments, shampooing, detangling treatments, scalp treatments, shine treatments, dandruff treatments, vitamin treatments, curling treatments, straightening treatments, permanent hair treatments, and temporary or permanent coloration treatments, and combinations thereof. An advantage of an embodiment can be that the silicone polymer protects the cuticle of the hair from surfactants, which can remove coloring agents such as dyes. For example, by protecting the cuticle of hair from surfactants, the color from a permanent of temporary coloring treatment can be preserved even after multiple washings with shampoo and soap. Another advantage of an embodiment of the method can be that the addition of body or weight to hair can facilitate the straightening of hair, or can prevent entanglement and frizzing. Another advantage of the method can be to protect the hair, skin, or nails from other forms of damage including physical, chemical and solar. The advantage of the embodiment of the method can provide for the directed expression of multiple attributes not previously feasible with simple application of silicone mixtures in the absence of covalent bonding, either as a mixture or in sequential application steps. The step of bringing can be from 1 second to 72 hours before or after a hair treatment. For nails, the bringing step should occur after nail color is applied without a period of intervening days to avoid chipping of nails or nail color.

Another method of covalently bonding a silicone polymer to hair or nails provides at least one aldehyde functionalized silicone polymer; brings the at least one aldehyde functionalized silicone polymer and a reactive composition into contact with the hair or nails; forming a covalent bond between the at least one aldehyde functionalized silicone polymer and hair or nails. The reactive composition can be a solution comprising water and at least one acid. The solution has a pH of about 4 to about 9. The reactive composition is not particularly limited so long as the components are acceptable for use with hair, nails, and or skin without causing heath problems or irritation. The pH is not particularly limited so long as the aldehyde is capable of reacting with the amine on the silicone polymer to form an imine linkage. During at least part of the step of forming a covalent bond, heated air or a heated surface is applied to hair or nails from a heat source, heated air or a heated surface can be applied to hair or nails from a heat source, wherein temperature of the heated air can be from 30-60° C., including 35-60° C. and 37-60° C., and wherein the heated surface can have a temperature of 35° C.-246° C., including 54° C.-246° C. or 130° F. to 475° F.

The at least one aldehyde functionalized silicone polymer has a molecular weight of from about 250 g/mol to about 50,000 g/mol, and contains from about 5 to about 15 mole percent of an aldehyde group. The at least one aldehyde functionalized silicone polymer has a molecular weight of from about 4,000 g/mol to about 9,000 g/mol, contains from about 6 to about 7 mole percent of the aldehyde group.

In another method, the providing step comprises: providing an amine functionalized silicone polymer and a compound having at least two aldehyde groups; and reacting the amine functionalized silicone polymer with a least one aldehyde group of the compound having a least two aldehyde groups to form the at least one aldehyde functionalized silicone polymer. The compound having at least two aldehyde groups is a compound of Formula (IV):

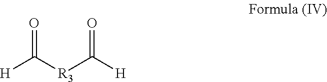

wherein R₃ is a (C₁-C₁₂) hydrocarbyl group, and wherein the hydrocarbyl group optionally includes at least one oxygen atom. The compound having at least two aldehyde groups is not particularly limited as long as at least two aldehyde groups are present on the same molecule. The compound having at least two aldehyde groups can be selected from the group consisting of: malonic dialdehyde, succinic dialdehyde, glutaraldehyde, adipaldehyde, 3-methyl glutaraldehyde, propyladipaldehyde, phthalic dialdehyde, terephthaldehyde, and malonic dialdehyde.

The method can be applied to any subject with hair or nail, including humans, but can also include animals having hair or nails. Further, the method can be applied to non-living subjects, including hair in wigs or clothing, such as coats, made from human or animal hair; artificial nails (e.g., acrylic nails); and artificial hair (e.g. polyesters, nylons, acrylic polymers), so long as the fake nails and hair are capable of undergoing a hydrosilylation reaction to produce a covalent bond to silicone.

In another embodiment, a method of treating is disclosed, wherein a person or animal in need of hair or nail treatment are treated to prevent or mitigate the effects of dryness, or flatness, or preventing color bleeding in a hair comprising: applying a composition comprising at least one photo-reactive silicone polymer; or a vinyl-containing silicone polymer to hair and a catalyst to the hair. The ingredients in the composition are not particularly limited, so long as the composition contains at least one photo-reactive silicone polymer or a vinyl-containing silicone polymer and a catalyst, which are capable of bonding the silicone polymer to hair. For example, the composition can comprise components known to the hair care industry, such as fragrances, additives, non-reactive polymers, and other ingredients typical of the hair care industry.

### EXAMPLES

### Example 1

### Synthesis of 4-Benzoyl Benzoic Acid Chloride From 4-Benzoyl Benzoic Acid

To a solution of 4-benzoyl benzoic acid (2.26 g, 10 mmol, Aldrich) in anhydrous 1,4-dioxane (20 mL) was added oxalyl chloride (1.76 mL, 20 mmol), followed by dimethylformamide (4 drops). After 45 minutes the mixture was concentrated to dryness under vacuum. The 4-benzoyl benzoic chloride acid produced was crystallized from 30 mL of 1:4 toluene/hexane. The solid 4-benzoyl benzoic chloride was rinsed with hexane and dried under vacuum to afford a white crystalline solid (1.26 g; melting point 92-92° C.).

### Example 2

### Synthesis of a Photo-Active Silicone Polymer From 4-Benzoyl Benzoic Acid Chloride and Aminopropyl Substituted Polydimethyl Siloxane

To a 1,4 dioxane (5 mL) solution of aminopropyl substituted polydimethylsiloxane (Gelest Inc., No. AMS-162, 3.35 g) was added 4-benzoylbenzoyl chloride (0.743 g) and diisopropylethylamine (0.574 mL). After stirring under nitrogen overnight, the mixture was diluted with tetrahydrofuran (10 mL) and washed with 1 N HCl (aq.) (6 mL). The 1 N HCl solution was back-extracted with diethyl ether (16 mL). The combined organic extracts were washed with 10% NaHCO₃, and then with brine. The solution was dried over magnesium sulfate, filtered, and concentrated under reduced pressure (room temperature, 0.2 Torr) to afford slightly cloudy yellow oil (3.92 g). Proton NMR showed complete conversion of the aminopropyl groups to amide groups to form a photo-active silicone polymer (4-benzyl benzoic acid-propyl-PDMS-amide, abbreviated BBA-propyl-PDMS-amide).

### Bonding of Silicones to Hair

The 4-benzyl benzoic-amide-PDMS was applied to hair samples or polyethylene. Unless otherwise note in Tables 1-4, the hair was treated with a composition containing 7.5 g of 4-benzyl benzoic-amide-PDMS, 20 ml of tetrahydrofuran, 10 ml of dioxane, and at least some of Hunig's base (N,N-diisopropylethylamine). Then the hair was dried in an oven at 115° C. before UV exposure. Unless otherwise noted, the UV light source was a Porta-Ray 400R 400 W metal halide arc lamp (Porta-Ray, Inc., Florida). The hair sample was then exposed to UV light from either a fluorescent bulb or the sun for times from 30 seconds to 1 hour as indicated in Table 1, below. A second washing was performed after 48 hours had lapsed since the previous washing. Additional changes to the procedures, if any, are as noted in the Tables below.

### Hair Sample Washing Procedure

The dry hair sample was massaged with LiQWd® volumizing shampoo for 2 minutes. The hair was rinsed with tap water. For a second or third wash, additional shampoo was applied to the wet hair, and the hair massaged with the shampoo for one minute. The hair was washed with tap water then with deionized water and dried after each wash. Additional changes to the procedures, if any, are as noted in the Tables below.

Analysis of Silicone Polymer after Washing

Qualitative elemental analysis was determined by Scanning Electron Microscope Energy Dispersive X-ray Analysis (SEM/EXDA). The SEM/EXDA number indicates a ratio of silicone to carbon for each sample measured. The higher the number shown, the more silicone that is present in the sample. Thus, a higher number indicates that higher amounts silicone polymers were retained in the hair after given treatment. Conversely, a lower number indicates that little or none of the photo-PDMS was retained. By inference, little or none of the photo-PDMS was bound to the hair. The retention or stability of the Si:C ratio after washing or repeated washings is interpreted to indicate that stable bonding of the photo-PDMS has occurred. Where there are no corresponding measurements, the area of the Table has been left blank.

TABLE 1
4-BBA-propyl-PDMS applied to polyethylene
SEM/EDXA analysis for silicone Si:C ratio

| | | 1x | 2x | 3x | Un- | |
|---|---|---|---|---|---|---|
| UV exposure | | wash | wash | wash | washed | Other Conditions |
| Dark | | 0 | | | | Untreated control |
| Dark | | 0 | | | | 4-BBA-propyl-PDMS |
| | | | | | | amide 5% solution |
| | | | | | | in THF |
| Dark | | 0.02 | | | | |
| Dark | | 0 | | | | |
| 30 | sec | 0.16 | 0.04 | | | 3 days between washes |
| 1 | min | 0.27 | 0.12 | | | 3 days between washes |
| 1 | min | 0.07 | | | | 5% |
| | | | | | | 4-BBA-propyl-PDMS |
| | | | | | | amide |
| 2 | min | 0.37 | 0.19 | | | 3 days between washes |
| None | | 0 | | | 0.61 | |
| 30 | min | n.a. | | 0.02, | | consecutive washes |
| | | | | 0.81 | | |
| 30 min, THF | | 0.24 | 0.03 | | | 3 h between washes |
| rinse | | | | | | |
| 30 min, 5% | | 0.39 | 0.10 | | | 3 h between washes |
| acetophenone | | | | | | |
| quench | | | | | | |
| 30 min, | | 0.76 | 0.44 | | | 3 h between washes, |
| sodium sulfite | | | | | | surface appeared |
| quench | | | | | | yellow and uneven |
| Dark | | 0 | | | | control sample. |
| | | | | | | 30 min before |
| | | | | | | washing |
| Dark | | 0 | | | | control sample. |
| | | | | | | 30 min before |
| | | | | | | washing |
| Fluorescent, | | 0 | | | | |
| 15 min | | | | | | |
| Fluorescent, | | 0 | | | | |
| 60 min | | | | | | |
| Sun, 15 minutes | | 0 | | | | |
| Sun, 15 minutes | | 0 | | | | |
| Sun, 1 hour | | 0.22 | | | | |
| Sun, 1 hour | | 0.40 | | | | |
| Sun, 1 hour | | 0.19 | | | | |
| Dark | | 0.00 | | | | 20% in THF |
| 2 | min | 0.21 | | | | 20% in THF |
| 2 | min | 0.29 | | | | 100% |
| 2 | min | 0.19 | | | | 4-BBA-propyl-PDMS |
| | | | | | | amide 772-76 |
| 30 | min | 0.02 | | | | 5% in THF |
| 30 | min | 0.23 | 0 | | | |
| 2 | min | 1.03 | | | | 4-BBA-propyl-PDMS |
| | | | | | | amide 772-103 |

Table 1 shows the bonding of the photo-PDMS to a test material (a polyethylene film). This testing was performed to confirm the activity of the photo-PDMS and to which parameters were important in bonding photo-PDMS to hair. This test establishes that hair treated with the photo-PDMS and no UV irradiation has a Si:C ratio of 0-0.02.

TABLE 2
20% 4-BBA-propyl-PDMS amide (AMS-162, 772-103A) applied to hair
SEM/EDXA analysis for silicone Si:C ratio

| | 1x | 2x | 3x | No | |
|---|---|---|---|---|---|
| UV exposure | wash | wash | wash | wash | Other Experimental Details |
| 30 min | 0.46 | | 0.24 | 3.54 | Wash 4 after two days |
| | | | | | Si:C = 0.28 |
| 30 min | 1.17 | 0.35 | | | second wash was 2 days |
| | | | | | after UV |
| 30 min | 0.16 | | | | wash was 2 days after UV |
| 2 min | 0.25 | 0.12 | | | second wash was 4 days |
| | | | | | after UV |

Table 2 shows the experimental conditions and results using photo-PDMS having 6-7 mole percent photo-reactive groups. The results demonstrate that PDMS is present in the hair even after two and three washings, and with UV exposure as short as 2 minutes.

All cited patents and publications referred to in this application are herein incorporated by reference in their entirety for all purposes.

What is claimed is:

1. A method of covalently bonding a silicone polymer to hair, or a nail comprising:
providing at least one photo-reactive silicone polymer,
bringing the at least one photo-reactive silicone polymer into contact with the hair or the nail, and
applying UV light from a light source to the at least one photo-reactive silicone polymer in the presence of the hair or the nail to produce a covalent bond between at least one silicone polymer and the hair or the nail,
wherein the UV light has a wavelength of from about 200 nm to about 400 nm.

2. The method of claim 1, wherein the at least one photo-reactive silicone polymer has a molecular weight of from about 250 g/mol to about 50,000 g/mol, and contains from about 5 to about 15 mole percent of a photo-reactive group.

3. The method of claim 2, wherein the at least one photo-reactive silicone polymer has a molecular weight of from about 4,000 g/mol to about 9,000 g/mol, contains from about 6 to about 7 mole percent of the photo-reactive group, and the photo-reactive group is represented by Formula (I): 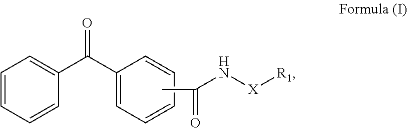
wherein the R₁ represents a silicone polymer, and X represents a linking group of a covalent bond or an alkyl group (CH₂)₁₋₁₀.

4. The method of claim 3, wherein the at least one photo-reactive silicone polymer has a molecular weight of from about 4,000 g/mol to about 6,000 g/mol, and the silicone polymer is a poly(dimethyl siloxane), and the photo-reactive group is represented by Formula (II): 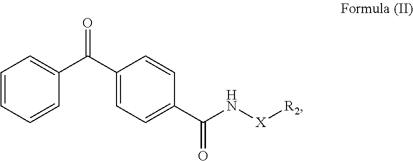
wherein R₂ represents the poly(dimethyl siloxane) and X represents a linking group of a covalent bond or an alkyl group (CH₂)₁₋₁₀.

5. The method of claim 1, wherein the bringing step comprises:
applying the at least one photo-reactive silicone polymer in a solution to the hair or the nail,
wherein the solution comprises an aprotic solvent.

6. The method of claim 1, wherein the providing step comprises:
synthesizing a compound represented by Formula (I) by reacting a compound represented by Formula (III): 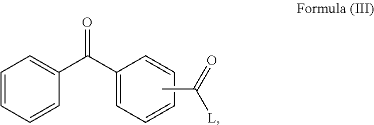
with an amine group attached to a silicone polymer to produce the compound represented by Formula (I) 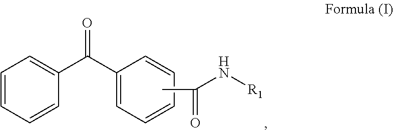
wherein R₁ represents a silicone polymer and L represents a leaving group.

7. The method of claim 6, wherein the leaving group is chloride.

8. The method of claim 7, wherein the synthesizing step comprises reacting a benzoyl benzoic acid with thionyl chloride or oxalyl chloride to produce the compound of Formula (III).

9. The method of claim 1, wherein, before or after the bringing step, the hair is subject to a permanent coloration, demi-permanent coloration, semi-permanent coloration, temporary hair color, or a straightening treatment or combination thereof.

10. A method of treating a hair to prevent or mitigate effects of dryness, or flatness, and/or preventing color bleeding in a hair comprising:
applying a composition comprising at least one photo-reactive silicone polymer to the hair.

11. The method of claim 10, wherein the at least one photo-reactive silicone polymer has a molecular weight of from about 250 g/mol to about 50,000 g/mol, and contains from about 5 to about 15 mole percent of a photo-reactive group.

12. The method of claim 11, wherein the at least one photo-reactive silicone polymer has a molecular weight of from about 4,000 g/mol to about 9,000 g/mol, contains from about 6 to about 7 mole percent of the photo-reactive group, and the photo-reactive group is represented by Formula (I): 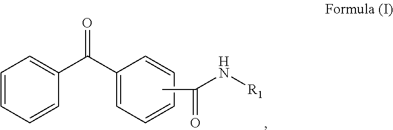
wherein the R₁ represents a silicone polymer.

13. The method of claim 10, wherein the composition comprises:
the at least one photo-reactive silicone polymer, wherein the composition comprises an aprotic solvent.

14. A method of covalently bonding a silicone polymer to hair, or a nail comprising:
providing at least one photo-reactive silicone polymer,
bringing the at least one photo-reactive silicone polymer into contact with the hair or the nail, and
applying UV light from a light source to the at least one photo-reactive silicone polymer in the presence of the hair or the nail to produce a covalent bond between at least one silicone polymer and the hair or the nail,
wherein the UV light has a wavelength of from about 200 nm to about 400 nm,
wherein the at least one photo-reactive silicone polymer has a molecular weight of from about 250 g/mol to about 50,000 g/mol, and contains from about 5 to about 15 mole percent of a photo-reactive group,
wherein the photo-reactive group is represented by Formula (V): 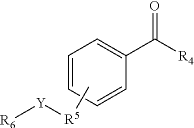
R₄ is selected from a (C₁-C₁₀) hydrocarbyl group;
R₅ is selected from the group consisting of: —N—(C═S)—N—; —(N—H)—; and —N═S(—H)—N—; —(C═O)—O—; —O—(C═O)—; and a (C₁-C₆) hydrocarbyl group, wherein the hydrocarbyl group is optionally substituted with one or more oxygen atoms, or hydroxyl groups; and
R₆ represents the silicone polymer; and
Y represents a linking group of a covalent bond or an alkyl group (CH₂)₁₋₁₀.

15. A method of bonding a silicone polymer to hair, or a nail comprising:
providing at least one photo-reactive silicone polymer,
bringing the at least one photo-reactive silicone polymer into contact with the hair or the nail, and
applying UV light from a light source to the at least one photo-reactive silicone polymer in the presence of the hair or the nail to bond at least one silicone polymer to the hair or the nail,
wherein the UV light has a wavelength of from about 200 nm to about 400 nm.

16. The method of claim 15, wherein the providing step comprises reacting a compound a benzoyl benzoic acid with a silicone polymer having at least one amine group to form a photo-reactive silicone polymer.

17. The method of claim 15, wherein the at least one photo-reactive silicone polymer has a molecular weight of from about 250 g/mol to about 75,000 g/mol, and contains from about 0.5 to about 50 mole percent of a photo-reactive group.

18. The method of claim 17, wherein the at least one photo-reactive silicone polymer has a molecular weight of from about 4,000 g/mol to about 60,000 g/mol, contains from about 1 to about 15 mole percent of the photo-reactive group, and the photo-reactive group is represented by Formula (I): 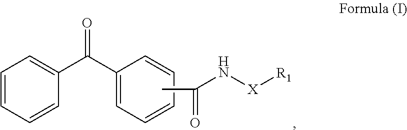
wherein the R₁ represents a silicone polymer, and X represents a linking group selected from the group consisting of: a covalent bond and a (C₁-C₁₀) hydrocarbyl group, optionally substituted with one or more of oxygen, nitrogen, sulfur, or silicon.

19. The method of claim 18, wherein the at least one photo-reactive silicone polymer has a molecular weight of from about 4,000 g/mol to about 50,000 g/mol, and the silicone polymer is a poly(dimethyl siloxane), and the photo-reactive group is selected from the group consisting of: 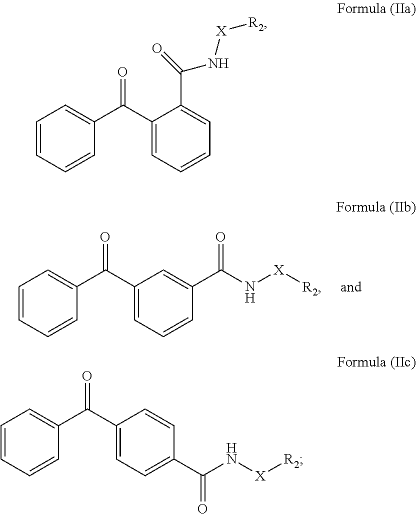
wherein R₂ represents the poly(dimethyl siloxane), and X represents a linking group selected from the group consisting of: a covalent bond and a (C₁-C₁₀) hydrocarbyl group, optionally substituted with one or more of oxygen, nitrogen, sulfur, or silicon.

20. The method of claim 15, wherein the bringing step comprises:
applying the at least one photo-reactive silicone polymer in a solution to the hair or the nail.

21. The method of claim 15, wherein the solution comprises an aprotic solvent.

22. The method of claim 15, wherein the providing step comprises:
reacting a compound represented by Formula (III): 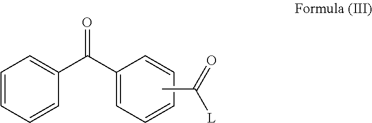
to produce the compound represented by Formula (I) 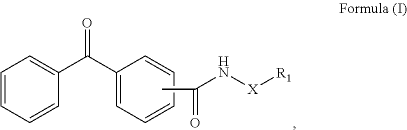
wherein R₁ represents a silicone polymer; X represents a linking group selected from the group consisting of: a covalent bond and a (C₁-C₁₀) hydrocarbyl group, optionally substituted with one or more of oxygen, nitrogen, sulfur, or silicon; and L represents Br or Cl.

23. The method of claim 15, further comprising a step of synthesizing a compound of Formula (III) by reacting a benzoyl benzoic acid with thionyl chloride, oxalyl chloride, or phosphorous tribromide to produce the compound of Formula (III).

24. The method of claim 15, wherein the providing step comprises:
reacting a compound of Formula (Ma); 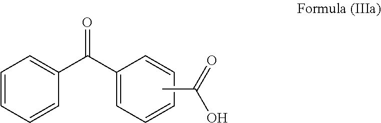
to form a compound of Formula (I); 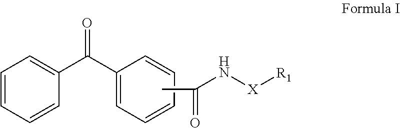
wherein R₁ represents a silicone polymer; and X represents a linking group selected from the group consisting of: a covalent bond and a (C₁-C₁₀) hydrocarbyl group, optionally substituted with one or more of oxygen, nitrogen, sulfur, or silicon.

25. The method of claim 15, wherein, before or after the bringing step, the hair is subject to a permanent coloration, demi-permanent coloration, semi-permanent coloration, temporary hair color, or a straightening treatment or combination thereof.

26. A method of bonding a silicone polymer to hair, or a nail comprising:
providing at least one photo-reactive silicone polymer,
bringing the at least one photo-reactive silicone polymer into contact with the hair or the nail, and
applying UV light from a light source to the at least one photo-reactive silicone polymer in the presence of the hair or the nail to bond at least one silicone polymer to the hair or the nail,
wherein the UV light has a wavelength of from about 200 nm to about 400 nm.

27. A method of applying a photo-reactive silicone polymer to hair or a nail comprising:
providing at least one photo-reactive silicone polymer, and
bringing the at least one photo-reactive silicone polymer into contact with the hair or the nail,
wherein the photo-reactive silicone polymer is capable of bonding to hair or a nail when the photo-reactive silicone polymer is exposed to UV light having a wavelength of from about 200 nm to about 400 nm from about 1 s to about 4 hours.
